(19) United States
(12) Patent Application Publication
(10) Pub. No.: US 2012/0204662 A1
(43) Pub. Date: Aug. 16, 2012

(54) Drive Mechanism and Drug Delivery Device

(75) Inventors: Claudia Matthias, Frankfurt am Main (DE); Steffen Raab, Frankfrut am Main (DE)

(73) Assignee: SANOFI-AVENTIS DEUTSCHLAND GMBH, Frankfurt am Main (DE)

(21) Appl. No.: 13/379,605

(22) PCT Filed: Jun. 28, 2010
(86) PCT No.: PCT/EP2010/059126

§ 371 Date: Apr. 18, 2012

(30) Foreign Application Priority Data
Jun. 30, 2009 (EP) 09008500.2

(51) Int. Cl. F16H 19/04; F16H 19/02

(52) U.S. Cl. 74/29; 74/25

(57) ABSTRACT

A drive mechanism (13) for a drug delivery device (1), comprising a housing (2), a transfer member (14), a drive member (16) and a piston rod (18). The transfer member (14) is configured to be rotated about a transfer member rotation axis (20) with respect to the housing (2) for delivering a dose of a drug (5). The drive member (16) is configured to be rotatable around a drive member rotation axis (39) with respect to the housing (2). The drive member (16) is arranged within the transfer member (14). When delivering the dose of the drug (5), the drive member (16) rotates about the drive member rotation axis (39). When delivering the dose of the drug (5), the drive member (16) and the transfer member (14) interact mechanically to convert rotation of the transfer member (14) about the transfer member rotation axis (20) into rotation of the drive member (16) about the drive member rotation axis (39), wherein rotation of the drive member (16) about the drive member rotation axis (39) is converted into translatory movement of the piston rod (18) with respect to the housing (2).

This disclosure relates to a drive mechanism for a drug delivery device and a drug delivery device incorporating such a drive mechanism.

A drug delivery device usually allows a user to set and deliver a dose of a drug.

In document EP 0204997 A1 a syringe drive apparatus is described comprising a hinged rack which is provided to drive a piston.

It is an object of the present disclosure to provide for a drive mechanism facilitating provision of an improved drug delivery device, for example a device with good dose accuracy as well as a device offering the possibility to deliver small doses of a drug. Furthermore, an improved drug delivery device is provided for.

This object may be achieved by a drive mechanism according to the independent claim. Further features are the subject matter of the dependent claims.

According to one aspect a drive mechanism for a drug delivery device is provided. The drug delivery device comprises a housing. The device further comprises a transfer member. The device has a drive member. The drug delivery device comprises a piston rod. The transfer member may be configured to be rotated about a transfer member rotation axis with respect to the housing for delivering a dose of a drug. The drive member may be configured to be rotatable about a drive member rotation axis with respect to the housing. The drive member may be arranged within the transfer member. When delivering the dose of the drug, the drive member may rotate about the drive member rotation axis. When delivering the dose of the drug, the drive member and the transfer member may interact mechanically to convert rotation of the transfer member about the transfer member rotation axis into rotation of the drive member about the drive member rotation axis, wherein rotation of the drive member about the drive member rotation axis is converted into translatory movement of the piston rod with respect to the housing.

One aspect relates to a drug delivery device comprising the above described drive mechanism. The device further comprises a cartridge holding a plurality of doses of the drug. The cartridge may be attached, permanently or releasably, to the housing of the drug delivery device.

The drug delivery device may comprise a cartridge holding a plurality of doses of a drug. Preferably, the drug is liquid medication, such as long-acting or short-acting insulin, heparin or growth hormones. The drug delivery device may be a pen-type device, e.g. a pen-type injector.

Mechanical interaction of the transfer member and the drive member, for example via engagement, may convert rotation of the transfer member about the transfer member rotation axis with respect to the housing into rotation of the drive member about the drive member rotation axis when delivering the dose of the drug. Rotation of the drive member about the drive member rotation axis may further be converted into translatory movement of the piston rod with respect to the housing due to mechanical interaction of the drive member and the piston rod. Preferably, the transfer member rotation axis extends axially along the housing of the drug delivery device, in particular along the main longitudinal axis of the housing. The drive member rotation axis may extend transversally, in particular perpendicularly, with respect to the main longitudinal axis of the housing.

The transfer member may be a transfer sleeve. With the drive member being arranged inside the transfer member a space-saving and reliably working drive mechanism may be achieved.

With the drive mechanism comprising the transfer member, the piston rod and the drive member a drive mechanism having

only a small number of operating members is achieved making the drive mechanism less prone to errors and hence guaranteeing a long life-time of the drive mechanism and, in particular, the drug delivery device incorporating the drive mechanism. In addition, the small number of operating members makes the drive mechanism, and hence, the drug delivery device, easily operable providing an especially user-friendly drug delivery device.

According to an embodiment, the transfer member may comprise an inner thread. The drive member may comprise a first gear. The first gear of the drive member may be configured to engage the inner thread of the transfer member.

Preferably, the first gear of the drive member is a gear wheel. Preferably, the first gear comprises a plurality of teeth. Rotational movement of the drive member may be achieved by the engagement of drive member and transfer member. Due to said engagement rotational movement of the transfer member may be converted into rotational movement of the drive member with respect to the housing when delivering the dose of the drug.

According to an embodiment, the drive member may comprise a second gear. When delivering the dose of the drug rotation of the second gear of the drive member about the drive member rotation axis with respect to the housing may be converted into translatory movement of the piston rod with respect to the housing.

Preferably, the second gear of the drive member may be a gear wheel. The second gear of the drive member may be coupled to the piston rod, preferably engaged with the piston rod, so as to convert rotational movement of the second gear about the drive member rotation axis with respect to the housing into translatory movement of the piston rod with respect to the housing, in particular movement of the piston rod in the distal direction with respect to the housing, for dispensing the dose of the drug. According to an embodiment, the first gear and the second gear of the drive member may be rotationally locked with respect to each other. Alternatively, the first gear and the second gear of the drive member may be formed unitarily.

Mechanical cooperation of the two gears, for example interlocking, may prevent relative rotational movement between the first gear of the drive member and the second gear of the drive member when delivering the dose of the drug.

According to an embodiment, the transfer member and the drive member may be secured against translatory movement with respect to the housing.

Preferably, interlocking of the transfer member and the housing as well as interlocking of the drive member and the housing prevents linear displacement of the transfer member and the drive member with respect to the housing.

According to an embodiment, the transfer member may comprise a first section and a second section. A diameter of the first section of the transfer member may be greater than a diameter of the second section of the transfer member. The piston rod may be arranged to be guided through the first section of the transfer member when delivering the dose of the drug.

The first section of the transfer member and the second section of the transfer member are expediently different from each other. The inner thread of the transfer member may be arranged in the first section of the transfer member. This may enable engagement of the first section of the transfer member with the first gear of the drive member such that rotational movement of the transfer member may be converted into rotational movement of the drive member with respect to the housing when delivering the dose of the drug. The drive member may be arranged in the section of the transfer member comprising the inner thread. Preferably, the piston rod may run through at least the first section of the transfer member when delivering the dose of the drug. With the piston rod being arranged in the transfer member a space-saving arrangement for the drive mechanism may be achieved.

According to an embodiment, the piston rod may comprise teeth. The second gear of the drive member may comprise a plurality of teeth. The teeth of the second gear of the drive member may be configured to engage the teeth of the piston rod for converting rotation of the second gear of the drive member about the drive member rotation axis into translatory movement of the piston rod with respect to the housing.

Mechanical interaction of the piston rod and the second gear of the drive member, preferably engagement, may convert rotational movement of the second gear of the drive member about the drive member rotation axis with respect to the housing into distal movement of the piston rod with respect to the housing for dispensing the dose of the drug.

According to an embodiment, the diameter of the first gear of the drive member is different from the diameter of the second gear of the drive member.

In this way, the first gear and the second gear may be adapted to linearly displace the piston rod only minimally for dispensing a minimal dose of the drug, i.e. a small dose which can still be delivered reliably by way of the drive mechanism.

According to another embodiment, the drive mechanism may comprise a dose member. The dose member may be configured to be displaced axially with respect to the housing when setting the dose of the drug. The dose member may be rotated with respect to the housing when delivering the dose of the drug. The transfer member may mechanically cooperate with the dose member. The transfer member may follow rotational movement of the dose member about the transfer member rotation axis with respect to the housing when delivering the dose of the drug.

Preferably, the transfer member and the dose member may be coupled, for example splined, such that rotational movement of the dose member about the transfer member rotation axis with respect to the housing when delivering the dose may be converted into rotational movement of the transfer member about the transfer member rotation axis with respect to the housing.

According to another embodiment, the drive mechanism may comprise a dose dial member. The dose dial member may be configured to be rotated with respect to the housing and with respect to the dose member when setting the dose of the drug. The dose dial member may be configured to be rotated in an opposite direction with respect to the housing when delivering the dose of the drug. The dose dial member may be rotated about the transfer member rotation axis with respect to the housing.

Preferably, when delivering the dose of the drug, there is mechanical cooperation between the dose dial member and the dose member, such that rotational movement of the dose dial member about the transfer member rotation axis with respect to the housing may be converted into rotational movement of the dose member about the transfer member rotation axis with respect to the housing when delivering the dose of the drug. Preferably, when setting the dose of the drug, mechanical cooperation between the dose dial member and the dose member may be limited or prevented, such that the dose dial member may rotate about the transfer member rotation axis and with respect to the dose member when the dose of the drug is set.

According to an embodiment, the drive mechanism may comprise a clutch mechanism. The clutch mechanism may comprise a first clutch member and a second clutch member. The first clutch member and the second clutch member may be adapted to cooperate mechanically with each other when delivering the dose of the drug, thereby rotationally locking the dose member and the dose dial member.

The clutch mechanism may provide mechanical cooperation of the first clutch member and the second clutch member only when delivering the dose. The first clutch member may be the dose member or a respective clutch member secured thereto. The second clutch member may be the dose dial member or a respective clutch member secured thereto. Preferably, when delivering the dose, the dose member and dose dial member both rotate about the transfer member rotation axis.

According to an embodiment, the clutch mechanism may comprise a spring. The spring may be configured to prevent mechanical cooperation of the first clutch member and the second clutch member when setting the dose of the drug, such that rotation of the dose member about the transfer member rotation axis when setting the dose of the drug may be prevented. For example, the spring may be biased to keep the clutch members out of engagement during dose setting.

According to an embodiment, at least one of the transfer member, the dose dial member and the dose member may rotate about the transfer member rotation axis when delivering the dose of the drug.

Preferably, there is mechanical interaction of the transfer member, the dose dial member and the dose member, for example splining or clutch engagement. Due to the mechanical interaction there may be common rotation of the transfer member, the dose dial member and the dose member about the transfer member rotation axis with respect to the housing when delivering the dose.

Further features and refinements become apparent from the following description of the exemplary embodiments in connection with the accompanying figures.

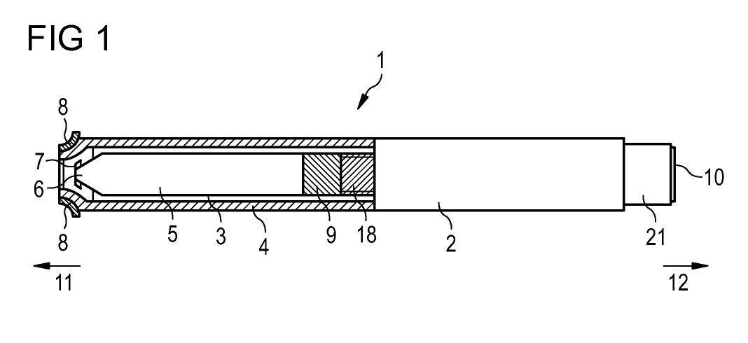

FIG. 1 schematically shows a partly sectional side view of an exemplary embodiment of a drug delivery device,

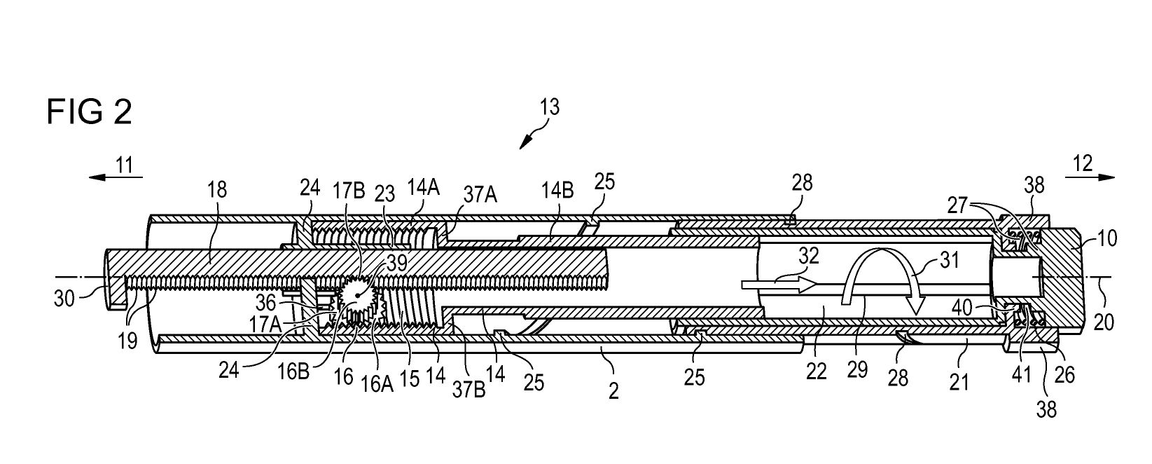

FIG. 2 schematically shows a sectional side view of an exemplary drive mechanism after or during setting of a dose of a drug,

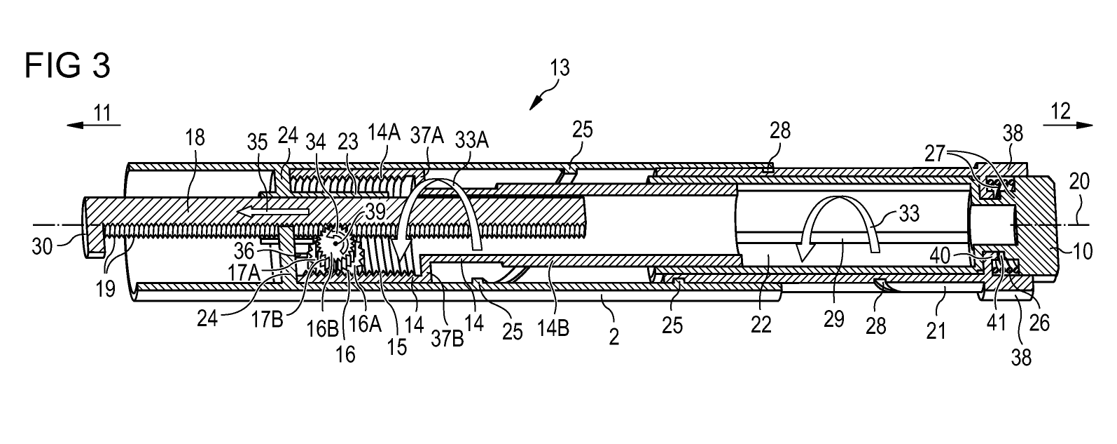

FIG. 3 schematically shows a sectional side view of the exemplary drive mechanism of FIG. 2 during delivery of the dose of the drug.

Like elements, elements of the same kind and identically acting elements may be provided with the same reference numerals in the figures.

In FIG. 1 a drug delivery device 1 is shown comprising a housing 2. Housing 2 may be an outer housing. The drug delivery device 1 comprises a cartridge 3 which is either permanently or releasably retained within a cartridge holder 4. The cartridge 3 contains a drug 5, preferably a plurality of doses of the drug 5. The drug 5 is preferably a liquid medication, for example comprising insulin, like short-acting or long-acting insulin, heparin or growth hormones.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)₄-des Pro36, des Pro37 Exendin-4(1-39)-NH2,
H-(Lys)₅-des Pro36, des Pro37 Exendin-4(1-39)-NH2,
des Pro36[Asp28] Exendin-4(1-39),
des Pro36[IsoAsp28] Exendin-4(1-39),
des Pro36[Met(O)14, Asp28] Exendin-4(1-39),
des Pro36[Met(O)14, IsoAsp28] Exendin-4(1-39),
des Pro36[Trp(O2)25, Asp28] Exendin-4(1-39),
des Pro36[Trp(O2)25, IsoAsp28] Exendin-4(1-39),
des Pro36[Met(O)14 Trp(O2)25, Asp28] Exendin-4(1-39),
des Pro36[Met(O)14 Trp(O2)25, IsoAsp28] Exendin-4(1-39); or
des Pro36[Asp28] Exendin-4(1-39),
des Pro36[IsoAsp28] Exendin-4(1-39),
des Pro36[Met(O)14, Asp28] Exendin-4(1-39),
des Pro36[Met(O)14, IsoAsp28] Exendin-4(1-39),
des Pro36[Trp(O2)25, Asp28] Exendin-4(1-39),
des Pro36[Trp(O2)25, IsoAsp28] Exendin-4(1-39),
des Pro36[Met(O)14 Trp(O2)25, Asp28] Exendin-4(1-39),
des Pro36[Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
H-(Lys)6-des Pro36[Asp28] Exendin-4(1-39)-Lys6-NH2,
des Asp28 Pro36, Pro37, Pro38Exendin-4(1-39)-NH2,
H-(Lys)6-des Pro36, Pro38[Asp28] Exendin-4(1-39)-NH2,
H-Asn-(Glu)5des Pro36, Pro37, Pro38[Asp28] Exendin-4(1-39)-NH2,
des Pro36, Pro37, Pro38[Asp28] Exendin-4(1-39)-(Lys)6-NH2,
H-(Lys)6-des Pro36, Pro37, Pro38[Asp28] Exendin-4(1-39)-(Lys)6-NH2,
H-Asn-(Glu)5-des Pro36, Pro37, Pro38[Asp28] Exendin-4(1-39)-(Lys)6-NH2,
H-(Lys)6-des Pro36[Trp(O2)25, Asp28] Exendin-4(1-39)-Lys6-NH2,
H-des Asp28 Pro36, Pro37, Pro38[Trp(O2)25] Exendin-4(1-39)-NH2,
H-(Lys)6-des Pro36, Pro37, Pro38[Trp(O2)25, Asp28] Exendin-4(1-39)-NH2,
H-Asn-(Glu)5-des Pro36, Pro37, Pro38[Trp(O2)25, Asp28] Exendin-4(1-39)-NH2,
des Pro36, Pro37, Pro38[Trp(O2)25, Asp28] Exendin-4(1-39)-(Lys)6-NH2,
H-(Lys)6-des Pro36, Pro37, Pro38[Trp(O2)25, Asp28] Exendin-4(1-39)-(Lys)6-NH2,
H-Asn-(Glu)5-des Pro36, Pro37, Pro38[Trp(O2)25, Asp28] Exendin-4(1-39)-(Lys)6-NH2,
H-(Lys)6-des Pro36[Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38[Met(O)14, Asp28] Exendin-4(1-39)-NH2,
H-Asn-(Glu)5-des Pro36, Pro37, Pro38[Met(O)14, Asp28] Exendin-4(1-39)-NH2,
des Pro36, Pro37, Pro38[Met(O)14, Asp28] Exendin-4(1-39)-(Lys)6-NH2,
H-(Lys)6-des Pro36, Pro37, Pro38[Met(O)14, Asp28] Exendin-4(1-39)-(Lys)6-NH2,
H-Asn-(Glu)5 des Pro36, Pro37, Pro38[Met(O)14, Asp28] Exendin-4(1-39)-(Lys)6-NH2,
H-Lys6-des Pro36[Met(O)14, Trp(O2)25, Asp28] Exendin-4(1-39)-Lys6-NH2,
H-des Asp28 Pro36, Pro37, Pro38[Met(O)14, Trp(O2)25] Exendin-4(1-39)-NH2,
H-(Lys)6-des Pro36, Pro37, Pro38[Met(O)14, Asp28] Exendin-4(1-39)-NH2,
H-Asn-(Glu)5-des Pro36, Pro37, Pro38[Met(O)14, Trp(O2)25, Asp28] Exendin-4(1-39)-NH2,
des Pro36, Pro37, Pro38[Met(O)14, Trp(O2)25, Asp28] Exendin-4(1-39)-(Lys)6-NH2,
H-(Lys)6-des Pro36, Pro37, Pro38[Met(O)14, Trp(O2)25, Asp28] Exendin-4(S1-39)-(Lys)6-NH2,
H-Asn-(Glu)5-des Pro36, Pro37, Pro38[Met(O)14, Trp(O2)25, Asp28] Exendin-4(1-39)-(Lys)6-NH2;
   or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The cartridge 3 has an outlet 6 and a membrane 7. The drug 5 can be dispensed from the cartridge 3 through the outlet 6. The drug delivery device 1 further comprises attaching means 8, preferably for attaching a needle assembly (not shown) to the cartridge holder 4. The drug delivery device 1 comprises a dose dial member 21 and a dose button 10. The dose button 10 may be part of a dose member (see dose member 22 in FIG. 2) or may be a separate member. In the latter case the dose button 10 may be secured to the dose member 22, in particular secured against rotational movement with respect to the dose member 22. The device 1 comprises a piston rod 18 and a piston 9.

The drug delivery device 1 and the housing 2 have a distal end and a proximal end. The distal end of the device 1 is indicated by arrow 11, which refers to that end of the drug delivery device 1 which is closest to a dispensing end of the drug delivery device 1. The proximal end of the device 1 is indicated by arrow 12 referring to that end of the device 1 which is furthest away from the dispensing end of the device 1.

The drug delivery device 1 may be a pen-type device, in particular a pen-type injector. The device 1 may be a disposable or a reusable device and may be configured to dispense fixed doses of the drug 5 or variable, preferably user-settable doses of the drug 5. The drug delivery device 1 may comprise a needle assembly (not shown in FIG. 1), comprising for example a needle covered by a needle mount, a needle retainer and a needle seal. The needle assembly may be, preferably releasably, attached to the distal end of the cartridge holder 4 or may be releasably attached to the attaching means 8, for example snap-fit elements. Alternatively, the drug delivery device 1 may be a needle-free device. In this case the attaching means 8 may be dispensed with.

The housing 2 has a proximal end and a distal end. The housing 2 may be designed to enable a safe and comfortable handling of the drug delivery device 1. The housing 2 may be configured to house, fix, protect and guide inner components of the drug delivery device 1 (e.g. cartridge holder 4, piston 9, piston rod 18, drive mechanism 13 (see FIGS. 2 and 3)). Preferably, the housing 2 limits or prevents the exposure to contaminants such as liquid, dirt or dust. The housing 2 may be an unitary or a multipart component. The housing 2 may comprise a tubular or cylindrical shape, as shown in FIG. 1. Alternatively, the housing 2 may comprise a non-tubular shape.

The cartridge holder 4 may be—permanently or releasably—attached, preferably glued or screwed, to the housing 2 of the drug delivery device 1. The cartridge 3 is, preferably releasably, retained within the cartridge holder 4. The cartridge holder 4 stabilizes the cartridge 3 mechanically. Additionally or alternatively, the cartridge holder 4 may be provided with a fixing member (not explicitly shown in FIG. 1), for example snapping means, for securing, preferably releasably securing, the cartridge 3 to the cartridge holder 4. A cartridge 3, which is releasably secured to the cartridge holder 4 may be detached from the cartridge holder 4, thereby allowing for a replacement cartridge 3 to be introduced into the cartridge holder 4, e.g. if all of the doses of the drug 5 that once were in the cartridge 3 have already been dispensed.

The outlet 6 may be covered by the membrane 7 protecting the drug 5 from external influences during storage of the cartridge 3. For delivery of the drug 5 the membrane 7 may be penetrated, for example pierced. The membrane 7 may be pierced by the needle assembly mentioned further above.

The piston 9 is retained within the cartridge 3. The piston 9 preferably comprises a resilient material. The piston 9 is movable with respect to the cartridge 3. The piston 9 may seal the cartridge 3 proximally. Movement of the piston 9 in the distal direction with respect to the cartridge 3 causes the drug 5 to be dispensed from the cartridge 3 through the outlet 6.

The piston rod 18 may operate through the housing 2 of the drug delivery device 1. The piston rod 18 may be designed to transfer axial movement through the drug delivery device 1, for example for the purpose of dispensing the drug 5 (see FIGS. 2 and 3 for more details). The piston rod 18 may be made of a flexible or a rigid material. The piston rod 18 may have a circular or a non-circular cross-section. The piston rod 18 may be of unitary or multipart construction.

The drug delivery device 1 comprises the dose dial member 21 and the dose button 10. Operation of the dose dial member 21 and the dose button 10 is described later on in connection with the description of FIG. 2 and FIG. 3. The drug delivery device 1 further comprises a drive mechanism 13 (not shown in FIG. 1) for setting and delivering a dose of the drug 5. The drive mechanism 13 is described in connection with FIGS. 2 and 3.

The drug delivery device 1 may be a manually, in particular a non-electrically, driven device. A force causing the dose dial member 21 to be moved, e.g. to be rotated and displaced axially, with respect to the housing 2 when delivering a dose of the drug 5 may be transferred to the piston rod 18 by means of the drive mechanism 13. Preferably, the force may be applied by a user.

FIG. 2 shows a drive mechanism 13 which is suitable to be implemented in the drug delivery device 1 (not explicitly shown in FIG. 2) as described previously. FIG. 2 shows the drive mechanism 13 after or during setting a dose of the drug 5.

The drive mechanism 13 presented in the following may for example be configured for setting and delivering doses of 30 IU or greater, for example a dose of 50 IU or greater, thereby providing high dose accuracy. Alternatively, the drive mechanism 13 may be designed for doses of 5 IU or less, preferably 1 IU or less, while having good dose accuracy.

The drive mechanism 13 is arranged within the housing 2 of the drug delivery device 1. The drive mechanism 13 comprises a transfer member 14. The transfer member 14 comprises a first section 14A and a second section 14B. The drive mechanism 13 comprises a drive member 16. The drive member 16 comprises a first gear 16A having a plurality of teeth 17A and a second gear 16B having a plurality of teeth 17B. The first gear 16A and the second gear 16B may be a gear wheel, respectively. The teeth 17A of the first gear 16A may be different from the teeth 17B of the second gear 16B. In particular, the number of teeth 17A of the first gear 16A may be different from the number of the teeth 17B of the second gear 16B. Additionally or accordingly, the spacing of teeth 17A of the first gear 16A may be different from the spacing of the teeth 17B of the second gear 16B. The drive member 16 is arranged inside the transfer member 14.

The drive mechanism 13 further comprises the piston rod 18 having teeth 19. The piston rod 18 comprises a shoe at its distal end. The shoe is expediently adapted to contact the piston. The drive mechanism is provided with a dose member 22 and a clutch mechanism 27. The drive mechanism 13 comprises the dose dial member 21 already mentioned in connection with the description of FIG. 1.

The transfer member 14 comprises the two sections, i.e. first section 14A and second section 14B, wherein the first section 14A has in the present embodiment a greater diameter than the second section 14B of the transfer member 14. In this embodiment, the first section 14A comprises an inner thread 15. Additionally or alternatively, the inner thread 15 may be arranged within the second section 14B of the transfer member 14.

The transfer member 14 may be secured against translatory movement with respect to the housing 2. The transfer member 14 may be secured against translatory movement in the distal direction with respect to the housing 2 by means of a distal stop member 24. Distal stop member 24 may be part of the housing 2 or an insert thereof. Additionally or alternatively, distal stop member 24 may be connected to the housing 2. Distal stop member 24 may protrude radially inwardly. Distal stop member 24 may be arranged circumferentially inside the housing 2 for preventing the movement of the transfer member 14 in the distal direction with respect to the housing 2. The transfer member 14 may be secured against translatory movement in the proximal direction with respect to the housing 2 by a respective proximal stop member (not explicitly shown in FIG. 2). The proximal stop member may be arranged at the proximal end of the first section 14A of the transfer member 14 extending circumferentially around this section within housing 2. At the proximal end of the first section 14A the transfer member 14 comprises a first shoulder 37A and a second shoulder 37B. In particular, the respective shoulder 37A and 37B is arranged at the transition between the first section 14A and the second section 14B extending radially inwardly with respect to the first section 14A. The respective shoulder 37A and 37B extends transversally with respect to a main longitudinal axis of the housing 2. The respective shoulder 37A and 37B may abut the proximal stop member for preventing movement of the transfer member 14 in the proximal direction with respect to the housing 2. Shoulders 37A and 37B may be separate shoulders or parts of a single shoulder.

The transfer member 14 may be coupled to the dose member 22 at the proximal end of the transfer member 14. In particular, the second section 14B of the transfer member 14 may be coupled to the dose member 22. Preferably, the dose member 22 is engaged with the transfer member 14. The transfer member 14 may be splined to the dose member 22, for example by means of a guide rib 29 of the dose member 22 and a corresponding guide notch (not explicitly shown) of the transfer member 14. Preferably, the guide rib 29 extends inside the dose member 22 along the main longitudinal axis of the housing 2. The corresponding guide notch may extend at an outer side of the transfer member 14, in particular at an outer side of the second section 14B of transfer member 14, along the main longitudinal axis of the housing 2. The splined connection of the dose member 22 and the transfer member 14 may prevent relative rotational movement of the dose member 22 with respect to the transfer member 14 and vice versa. Transfer member 14 and dose member 22 may be permanently rotationally locked.

The transfer member 14 is configured to be rotatable with respect to the housing 2. The transfer member 14 may rotate about a transfer member rotation axis 20. Preferably, the transfer member 14 rotates with respect to the housing 2 only when delivering the dose of the drug 5 as explained later in connection with the description of the operation of the drive mechanism 13. The transfer member 14 does not rotate with respect to the housing 2 when setting the dose of the drug 5.

The transfer member rotation axis 20 may extend through the transfer member 14, the dose member 22 and the dose dial member 21. The transfer member rotation axis 20 may be the main longitudinal axis of the housing 2.

The piston rod 18 may be arranged to run through the first section 14A of the transfer member 14, which expediently comprises the inner thread 15, when delivering a dose of the drug 5. Preferably, the piston rod 18 runs through the first section 14A and the second section 14B of the transfer member when delivering the dose of the drug 5.

The piston rod 18 is configured to move only linearly with respect to the housing 2. The piston rod 18 is configured to be not rotatable with respect to the housing 2. The piston rod 18 moves in the distal direction with respect to the housing 2 when delivering the dose. The piston rod 18 does not move in the proximal direction with respect to the housing 2 when setting the dose. The piston rod 18 may be prevented from rotational movement with respect to the housing 2 when setting and when delivering the dose of the drug 5.

The piston rod 18 comprises teeth 19. The piston rod 18 may be a gear rod, for example. A gear rod may have the advantage that a further bearing member (not explicitly shown), arranged between the piston rod 18 and the piston 9 for abutting a proximal end of the piston 9 and hence, advancing the piston 9, may be redundant.

The piston rod 18 may be axially guided when being moved through the transfer member 14 with respect to the housing 2 when delivering the dose of the drug 5 by means of a guide rib 23. Guide rib 23 may be part of the housing 2, preferably guide rib 23 may be integrally formed with the housing 2. Alternatively, guide rib 23 may be connected to the housing 2. Guide rib 23 may extend along the main longitudinal axis of the housing 2, preferably shifted radially inwardly from the inner surface of the housing 2.

The piston rod 18 may comprise a surface 30 at the distal end of the piston rod 18 arranged to interact with the piston 9. Surface 30 may extend transversally with respect to the main longitudinal axis of the housing 2. Surface 30 may be part of the shoe the piston rod 18 is provided with at its distal end. When delivering the dose, the piston rod 18 may be moved in the distal direction with respect to the housing 2 as explained later on in more detail. Thereby, the surface 30 of the piston rod 18 may abut piston 9. When moved in the distal direction with respect to the housing 2, the piston rod 18 pushes piston 9 in the distal direction with respect to the housing 2 and with respect to the cartridge 3 (not shown in FIG. 2), thereby dispensing the dose of the drug 5.

The drive member 16 comprises the first gear 16A and the second gear 16B, wherein the first gear 16A and the second gear 16B are preferably rotationally locked with each other, which means that the first gear 16A cannot rotate with respect to the second gear 16B and vice versa. The first gear 16A and the second gear 16B of the drive member 16 may be unitary. A diameter of the first gear 16A of the drive member 16 may be different from a diameter of the second gear 16B of the drive member 16.

Preferably, the first gear 16A, i.e. the gear interacting with the transfer member 14, comprises the larger diameter. Additionally or alternatively, the first gear 16A and the second gear 16B may comprise equal diameters. The number of teeth 17A of the first gear 16A may be different from the number of teeth 17B of the second gear 16B of the drive member 16. The spacing between the teeth 17A of the first gear 16A may be different from the spacing between the teeth 17B of the second gear 16B of the drive member 16.

Drive member 16 may be arranged inside transfer member 14, thus providing a space-saving realization of the drive mechanism 13. Preferably, the drive member 16 and the transfer member 14 represent a worm gear involving expediently the inner thread 15. The teeth 17A of the first gear 16A of the drive member 16 may be arranged to engage the inner thread 15. The teeth 17B of the second gear of the drive member 16 may be arranged to engage the teeth 19 of the piston rod 18.

Preferably, the drive member 16 is secured against translatory movement with respect to the housing 2 and with respect to the transfer member 14, for example by means of gear support 36 attached to the housing 2. Additionally or accordingly, the gear support 38 may be attached to the transfer member 14.

Drive member 16 is configured to rotate with respect to the housing 2. Thereby, drive member 16, i.e. the first gear 16A and the second gear 16B of the drive member 16, may rotate about a drive member rotation axis 39, which may run transversally, preferably perpendicularly, with respect to the transfer member rotation axis 20, i.e. the main longitudinal axis of the housing 2 in this embodiment. Thereby, rotation of the transfer member 14 about the transfer member rotation axis 20 when delivering the dose of the drug 5 may be converted into rotation of the first gear 16A of the drive member 16 and hence, rotation of the second gear 16B due to the rotational locking of the first gear 16A and the second gear 16B, about the drive member rotation axis 39 when delivering the dose via teeth 17A of the first gear 16A of the drive member 16 engaging the inner thread 15 of the rotating transfer member 14. In this embodiment, the drive member 16 may rotate with respect to the housing 2 only when delivering the dose of the drug 5, as explained later in connection with the description of the operation of the drive mechanism 13.

Once the drive member 16 is rotating, e.g. when delivering the dose of the drug 5, rotation of the second gear 16B of the drive member 16 about the drive member rotation axis 39 may be converted into linear movement of the piston rod 18 in the distal direction with respect to the housing 2 when delivering the dose of the drug 5 via teeth 17B of the second gear 16B of the drive member 16 successively engaging the teeth 19 of the piston rod 18, thereby advancing the piston rod 18.

Due to the different diameters of the first gear 16A and the second gear 16B of the drive member 16 and, in particular due to the shape of the inner thread 15 the second gear 16B is engaging, the conversion of rotational movement of the dose dial member 21 or the transfer member into linear movement of the piston rod 18 with respect to the housing 2 when delivering the dose may be adapted to cause only marginal distal displacement of the piston rod 18 and hence, to cause dispensing of a small dose of the drug 5, while still providing high dose accuracy. Marginal distal displacement of the piston rod 18 may also be achieved due to the different number of teeth 17A and teeth 17B of the first gear 16A and the second gear 16B. Additionally or accordingly, marginal distal displacement of the piston rod 18 may be achieved due to the different spacing of teeth 17A and teeth 17B of the first gear 16A and the second gear 16B. For reducing the extent of displacement of the piston rod 18 in the distal direction with respect to the housing 2, the number of teeth 17B of the second gear 16B should be increased, i.e. may be greater than the number of teeth 17A of the first gear 16A, for example. Additionally or alternatively, the inner thread 15 of the transfer member 14 may be made less steep for reducing the extent of displacement of the piston rod 18 in the distal direction with respect to the housing 2. The piston rod 18 may be displaced in the distal direction with respect to the housing 2 along the transfer member rotation axis 20.

As the drive member 16 does not rotate when setting a dose of the drug 5, and correspondingly the first gear 16A of drive member 16 and second gear 16B of drive member 16 do not rotate with respect to the housing 2 when setting the dose of the drug 5, linear movement of the piston rod 18 in the proximal direction with respect to the housing 2 when setting the dose does not occur.

The drive mechanism 13 comprises the dose dial member 21. The dose dial member 21 may comprise a dose dial grip 38 arranged at the outer side at the proximal end of the dose dial member 21. Dose dial grip 38 may be gripped and rotated with respect to the housing 2 by a user for setting a dose of the drug 5. In particular, dose dial grip 38 may be rotated with respect to the dose button 10 when setting the dose to be delivered. Due to this embodiment an easily manageable and operable drug delivery device 1 may be obtained.

The dose dial member 21 may be designed as an insert sleeve within housing 2. The dose dial member 21 is movable with respect to the housing 2. The dose dial member 21 is preferably connected to the housing 2. The dose dial member 21 comprises a thread 28 arranged at the outer side of the dose dial member 21. A thread 25 is provided inside the housing 2 for a threaded engagement of the housing 2 and the dose dial member 21. Rotation of the dose dial member 21 thus results in axial movement of the dose dial member 21 with respect to the housing 2.

The dose dial member 21 may be rotated in a first direction with respect to the housing 2 for setting a dose of the drug 5. Thereby, the dose dial member 21 is moved axially, in particular in the proximal direction. The dose dial member 21 may be rotatable in a second direction with respect to the housing 2 during delivery of the dose of the drug 5. Thereby, the dose dial member 21 is moved axially, in particular in the distal direction. The second direction may be opposite to the first direction. Rotation of the dose dial member 21 may occur about the main longitudinal axis of the housing 2, for example.

The distance by which the dose dial member 21 is displaced axially with respect to the housing 2 when setting the dose of the drug 5 may determine a size of a dose of the drug 5. The proximal end position and the distal end position of the dose dial member 21 with respect to the housing 2 may be determined by a respective stop feature limiting the proximal and distal movement of the dose dial member 21 with respect to the housing 2.

The drive mechanism 13 comprises the dose member 22 which may be arranged at least partly within the dose dial member 21. The dose member 22 may be retained within the dose dial member 21. The dose member 22 comprises an indentation 41 at the proximal end of the dose member 22. In particular, the indentation 41 is arranged at the transition between dose member 22 to the dose button 10, which is arranged at the proximal end of the dose member 22. The indentation 41 comprises a distal end and a proximal end. The dose dial member 21 comprises an opening at the proximal end of the dose dial member 21. In particular, the dose button 10 protrudes from the opening of dose dial member 21 at the proximal end of the dose dial member 21. The dose button 10 may be part of the dose member 22 or may be a separate member connected to the dose member 22. Dose button 10 may be non-rotatable with respect to the dose member 22. Dose button 10 may be configured to be pushed by a user for dispensing the set dose of the drug 5 providing an easy operation of the drug delivery device 1.

In this embodiment, the dose dial member 21 and, in particular, the dose dial grip 38, may be rotated in a first direction with respect to the housing 2 and with respect to the dose member 22 when setting the dose of the drug 5. When setting the dose, i.e. when rotating the dose dial member 21 in the first direction, the dose dial member 21 moves proximally with respect to the housing 2 due to the threaded engagement of the dose dial member 21 with the housing 2. The dose dial member 21 and in particular, the dose dial grip 38, may be rotated in a second direction with respect to the housing 2 when delivering the dose of the drug 5, the second direction being opposite to the first direction. When delivering the dose the dose dial member 21 rotates and moves distally with respect to the housing until the dose dial member 21 abuts the dose member 22.

The dose dial member 21 comprises a shoulder 40, preferably in the proximal region. The shoulder 40 protrudes inwardly with respect to the dose dial member 21. A spring 26 may be integrated within the dose dial member 21 at the proximal end of the dose dial member 21. Alternatively, the spring may be a separate component. The shoulder 40 abuts the indentation 41 of the dose member 22, in particular, the shoulder 40 abuts the proximal or the distal end of the indentation 41. Due to mechanical interaction of the shoulder 40, the spring 26 and the distal or the proximal end of the indentation 41, the dose member 22 follows the axial movement of the dose dial member 21 when setting and when delivering the dose of the drug. This means that the dose dial member 21 pulls the dose member in the proximal direction with respect to the housing 2 when setting the dose. The dose dial member 21 pushes the dose member in the distal direction with respect to the housing 2 when delivering the dose. Thus, there is no relative axial movement between the dose member 22 and the dose dial member 21 during setting of the dose.

The dose member 22 and the dose dial member 21 may be releasably rotationally lockable by means of a clutch mechanism 27. Clutch mechanism 27 may comprise a first clutch member and a second clutch member. The first clutch member may be arranged at the dose member 22, for example. The second clutch member may be arranged at the dose dial member 21, for example. Alternatively, the first clutch member may be the dose member 22 and the second clutch member may be the dose dial member 21 or vice versa, for example. The clutch mechanism 27 comprises the spring 26. The spring keeps the clutch members out of engagement during dose setting.

When activating the clutch mechanism 27, for example by pushing dose button 10 of the dose member 22 in the distal direction with respect to the housing 2, the first clutch member, in this embodiment the dose member 22, in particular the dose button 10, may be marginally pushed in the distal direction with respect to the housing 2 and with respect to the second clutch member, in this embodiment the dose dial member 21. Dose member 22 and dose dial member 21 may abut. In particular, the proximal end of the indentation 41 arranged at the proximal end of the dose member 22 abuts the shoulder 40 of the dose dial member 21.

Thereby, the first clutch member and the second clutch member may cooperate mechanically. For example, teeth (not explicitly shown in FIG. 2) arranged at the first clutch member and at the second clutch member may cooperate mechanically, for example interlock, such that the dose member 22 and the dose dial member 21 are rotationally locked. If the dose button 10 is released, the spring 26 unlocks dose member 22 and dose dial member 21 from one another.

During rotational locking, rotational movement of the dose dial member 21 with respect to the housing 2 about the transfer member rotation axis 20 may be converted into rotational movement of the dose member 22 with respect to the housing 2 about the transfer member rotation axis 20. Activation of the clutch mechanism 27, e.g. by pushing onto the dose button 10 in this embodiment, preferably occurs only after the dose of the drug 5 has been set, i.e. when delivering the dose of the drug 5, as explained later on in more detail. For keeping the clutch mechanism 27 activated and hence, for keeping the dose member 22 and the dose dial member 21 rotationally locked, the user has to keep on pushing onto the dose button 10 while delivering the dose of the drug 5.

De-activation of the clutch mechanism 27 may for example occur when a user stops pushing onto dose button 10, preferably after the dose of the drug 5 has been delivered.

After having released the dose button 10, the spring 26 pushes automatically the first clutch member (in this embodiment the dose member 22, in particular the dose button 10) proximally with respect to the housing 2 such that the first clutch member and the second clutch member can no longer cooperate mechanically. Hence, the dose member 22 and the dose dial member 21 are no longer rotationally locked after the user has released the dose button 10.

In the following, operation of the drive mechanism 13 for setting a dose of the drug 5 from the cartridge 3 of FIG. 1 is described. FIG. 2 represents the state of the drive mechanism 13 after the dose has been set or during dose setting. In particular, FIG. 2 shows the dose dial member 21 and the dose member 22 after having already been moved proximally with respect to the housing 2. Operation of the drive mechanism 13 for delivering the dose of the drug 5 from the cartridge 3 of FIG. 1 is described in conjunction with the description of FIG. 3.

In this embodiment, when setting a dose of the drug 5, the dose dial member 21 is rotated with respect to the dose member 22. The clutch mechanism 27 is not activated, i.e. the dose button 10 may not be pushed by the user. Hence, the first clutch member and the second clutch member do not cooperate mechanically, i.e. they are not locked rotationally. This allows rotational movement of the dose dial member 21 with respect to the dose member 22 when setting the dose. The dose member 22 follows axial movement of the dose dial member 21, in particular due to interaction of the shoulder 40, the proximal or the distal end of the indentation 41 and the spring 26, as described previously. Thus, there is no relative axial movement between the dose member 22 and the dose dial member 21. Activation of the clutch mechanism 27 may occur after the dose has been set. In this embodiment, the clutch mechanism 17 may be activated by a user pushing onto the dose button 10. The dose button 10 may thus be moved in the distal direction with respect to the housing 2 and, in particular with respect to the dose dial member 21 until the clutch members interact.

To set a dose of the drug 5 a user may manually rotate the dose dial member 21, in particular the dose dial grip 38, in the first direction about the transfer member rotation axis 20 with respect to the housing 2, as indicated by arrow 31. In addition, the dose dial member 21 moves axially in the proximal direction with respect to the housing 2.

When setting the dose, the dose dial member 21 is not rotationally locked with the dose member 22, the dose dial member 21 thereby rotates with respect to the housing 2 and with respect to the dose member 22. Rotation of the dose dial member 21 about the transfer member rotation axis 20 in the dose setting direction is converted into linear movement of the dose member 22 in the proximal direction with respect to the housing 2, as indicated by arrow 32. Hence, the dose member 22 moves only axially when setting the dose. The dose member 22 is prevented from rotating when setting the dose of the drug 5.

As the dose member 22 does not rotate when setting the dose and the dose member 22 is splined to the transfer member 14, rotational movement of the transfer member 14 about the transfer member rotation axis 20 is prevented when setting the dose.

Hence, the drive member 16, which is driven by the transfer member 14, i.e. the first gear 16A which is adapted to be driven by the transfer member 14 via teeth 17A engaging the inner thread 15 of the transfer member 14, does not rotate about the drive member rotation axis 39 with respect to the housing 2 when setting the dose. As it is rotationally locked with the first gear 16A of the drive member 16, the second gear 16B of the drive member 16 does not rotate as well. Hence, as rotation of the drive member 16 is prevented when setting the dose, the piston rod 18 is prevented from being displaced linearly, i.e. the piston rod 18 is prevented from moving in the proximal direction with respect to the housing 2 when setting the dose of the drug 5. Hence, a good dose accuracy of the drug delivery device 1 may be achieved.

When the dose dial member 21 has been rotated by an angle corresponding to a desired dose of the drug 5, the dose may be dispensed. Additionally or accordingly, when the extent of proximal displacement of the dose dial member 21 with respect to the housing 2 corresponds to a desired dose of the drug 5, the dose may be dispensed. FIG. 3 shows the drive mechanism 13 of FIG. 2 during delivery of the dose of the drug 5.

Once the dose of the drug 5 has been set, the user may acticate the clutch mechanism 27, for example by pushing onto the dose button 10. Consequently, the dose member 22—in particular the dose button 10—is pushed in the distal direction with respect to the housing 2 and with respect to the dose dial member 21. Hence, the first clutch member and the second clutch member, e.g. in this embodiment the dose member 22 and the dose dial member 21, cooperate mechanically, in particular, engage with each other, such that the dose member 22 and the dose dial member 21 are releasably rotationally locked, as described previously.

After the user has pushed onto the dose button 10, i.e after the user has activated the clutch mechanism 27, the dose dial member 21 rotates in the second direction about the transfer member rotation axis 20 with respect to the housing 2, indicated by arrow 33, thereby moving in the distal direction with respect to the housing 2. The second direction of rotation is opposite to the first direction in which the dose dial member 21 was rotated when setting the dose of the drug 5.

During rotation of the dose dial member 21 in the second direction, i.e. during dose delivery, the user keeps on pushing onto the dose button 10 for keeping clutch mechanism 27 activated. Otherwise, if the user stopped pusing onto the dose button 10, spring 26 would push the dose member 22 in proximal direction, hence de-activating the clutch mechanism 27 causing the dose member 22 and the dose dial member 21 to be no longer rotationally locked.

When rotationally locked, rotation of the dose dial member 21 about the transfer member rotation axis 20 in the second direction with respect to the housing 2 is converted into rotation of the dose member 22 about the transfer member rotation axis 20 in the second direction with respect to the housing 2, indicated by arrow 33. In addition, the dose member 22 moves distally following distal movement of the dose dial member 21 with respect to the housing 2.

As the transfer member 14 is engaged with, preferably splined to, the dose member 22, rotation of the dose member 22 about the transfer member rotation axis 20 in the second direction with respect to the housing 2 is converted into rotation of the transfer member 14 about the transfer member rotation axis 20 in the second direction with respect to the housing 2 (see arrow 33A). Consequently, when delivering the dose, the dose dial member 21, the dose member 22 and the transfer member 14 rotate about the same rotation axis, i.e. transfer member rotation axis 20 in this embodiment.

When the transfer member 14 rotates, the transfer member 14 and the drive member 16 interact mechanically, i.e. the teeth 17A of the first gear 16A of the drive member 16 successively engage the inner thread 15 of the transfer member 14 and hence, rotation of the transfer member 14 about the transfer member rotation axis 20 in the second direction with respect to the housing 2 is converted into rotation of the drive member 16, i.e. the first gear 16A and the second gear 16B of the drive member 16, about the drive member rotation axis 39 with respect to the housing 2, wherein the drive member rotation axis 39 runs transversally with respect to the transfer member rotation axis 20. The rotation of the drive member 16 is indicated by arrow 34.

With the drive member 16, in particular the second gear 16B of the drive member 16, rotating about the drive member rotation axis 39 with respect to the housing 2 the teeth 17B of the second gear 16B engage successively with the teeth 19 of the piston rod 18, with rotational movement of the second gear 16B of the drive member 16 being converted into displacement of the piston rod 18 with respect to the housing 2 (see arrow 35). Hence, the piston rod 18 may displace the piston 9 with respect to the cartridge 3 (not shown in FIG. 3), thereby dispensing the dose of the drug 5 through the outlet 6 of the cartridge 3 (see FIG. 1). The piston rod 18 may be displaced in the distal direction with respect to the housing 2 along the transfer member rotation axis 20.

After having delivered the dose of the drug, the user releases the dose button 10, hence de-activating the clutch mechanism 27, as spring 26 pushes the dose member 22, in particular the dose button 10, in the proximal direction, preventing further mechanical cooperation of the dose member 22 and the dose dial member 21. Thus, setting of another dose of the drug 5 is enabled.

The drive mechanism 13 presented herein has the advantage of comprising only a small number of operating members (i.e. transfer member 14, drive member 16, piston rod 18, dose member 22 and dose dial member 21). This makes the drive mechanism 13 less prone to errors, hence, guaranteeing a long life-time of the drug delivery device 1.

In addition, due to a corresponding shaping of the inner thread 15 of the transfer member 14 dispensing of small doses with high dose accuracy may be achieved. Additionally or accordingly, dispensing of small doses with high dose accuracy may be achieved due to the different diameters of the first gear 16A and the second gear 16B of the drive member 16. Additionally or accordingly, due to a corresponding spacing of the teeth 17A and 17B of the drive member 16 dispensing of small doses with high dose accuracy may be achieved. Additionally or accordingly a corresponding number of teeth 17A and 17B may enable dispensing of small doses with high dose accuracy. Thereby, the drug delivery device 1 may be designed for dispensing doses of 5 IU or less, preferably of 1 IU or less with high dose accuracy. Alternatively, drug delivery device 1 may be used to deliver doses greater than 30 IU, for example a dose of 50 IU providing high dose accuracy.

Other implementations are within the scope of the following claims. Elements of different implementations may be combined to form implementations not specifically described herein.

### REFERENCE NUMERALS

1 Drug delivery device
2 Housing
3 Cartridge
4 Cartridge holder
5 Drug
6 Outlet
7 Membrane
8 Attaching means
9 Piston
10 Dose button
11 Distal end
12 Proximal end
13 Drive mechanism
14 Transfer member
14A Second section
14B First section
15 Thread
16 Drive member
16A First gear of drive member
16B Second gear of drive member
17A Teeth
17B Teeth
18 Piston rod
19 Teeth
20 Transfer member rotation axis
21 Dose dial member
22 Dose member
23 Guide rib
24 Stop member
25 Thread
26 Spring
27 Clutch mechanism
28 Thread
29 Guide rib
30 Surface
31 Arrow
32 Arrow
33 Arrow
33A Arrow
34 Arrow
35 Arrow
36 Gear support
37A First shoulder
37B Second shoulder
38 Dose dial grip
39 Drive member rotation axis
40 Shoulder
41 Indentation

1. A drive mechanism for a drug delivery device, comprising:
a housing,
a transfer member which is configured to be rotated about a transfer member rotation axis with respect to the housing for delivering a dose of a drug,
a drive member which is configured to be rotatable about a drive member rotation axis with respect to the housing, wherein the drive member is arranged within the transfer member,
a piston rod,
wherein, when delivering the dose of the drug, the drive member rotates about the drive member rotation axis and wherein, when delivering the dose of the drug, the drive member and the transfer member interact mechanically to convert rotation of the transfer member about the transfer member rotation axis into rotation of the drive member about the drive member rotation axis, wherein rotation of the drive member about the drive member rotation axis is converted into translatory movement of the piston rod with respect to the housing.

2. The drive mechanism according to claim 1, wherein the transfer member comprises an inner thread and wherein the drive member comprises a first gear, wherein the first gear of the drive member is configured to engage the inner thread of the transfer member.

3. The drive mechanism of claim 2, wherein the drive member comprises a second gear, wherein, when delivering the dose of the drug, rotation of the second gear of the drive member about the drive member rotation axis with respect to the housing is converted into translatory movement of the piston rod with respect to the housing.

4. The drive mechanism according to claim 3, wherein the first gear and the second gear of the drive member are rotationally locked with respect to each other or formed unitarily.

5. The drive mechanism according to claim 1, wherein the transfer member and the drive member are secured against translatory movement with respect to the housing.

6. The drive mechanism according to claim 1,
wherein the transfer member comprises a first section and a second section, a diameter of the first section of the transfer member being greater than a diameter of the second section of the transfer member and the piston rod being arranged to be guided through the first section of the transfer member when delivering the dose of the drug.

7. The drive mechanism according to claim 6, wherein the inner thread is arranged in the first section of the transfer member.

8. The drive mechanism according to claim 3, wherein the piston rod comprises teeth and wherein the second gear of the drive member comprises a plurality of teeth, with the teeth of the second gear of the drive member being configured to engage the teeth of the piston rod for converting rotation of the second gear of the drive member about the drive member rotation axis into translatory movement of the piston rod with respect to the housing.

9. The drive mechanism according to claim 8, wherein the diameter of the first gear of the drive member is different from the diameter of the second gear of the drive member.

10. The drive mechanism according to claim 1, wherein the drive mechanism comprises a dose member which is configured to be displaced axially with respect to the housing when setting the dose of the drug and to be rotated with respect to the housing when delivering the dose of the drug, wherein the transfer member mechanically cooperates with the dose member and the transfer member follows rotational movement of the dose member about the transfer member rotation axis with respect to the housing when delivering the dose of the drug.

11. The drive mechanism according to claim 10, wherein the drive mechanism comprises a dose dial member which is configured to be rotated with respect to the housing and with respect to the dose member when setting the dose of the drug and which is configured to be rotated in an opposite direction with respect to the housing when delivering the dose of the drug, wherein the dose dial member is rotated about the transfer member rotation axis with respect to the housing.

12. The drive mechanism according to claim 11, wherein the drive mechanism comprises a clutch mechanism comprising a first clutch member and a second clutch member, wherein the first clutch member and the second clutch member are adapted to cooperate mechanically with each other when delivering the dose of the drug, thereby rotationally locking the dose member and the dose dial member.

13. The drive mechanism of claim 10, wherein the clutch mechanism comprises a spring which is configured to prevent mechanical cooperation of the first clutch member and the second clutch member when setting the dose of the drug.

14. The drive mechanism according to claim 10, wherein the transfer member, the dose dial member and the dose member rotate about the transfer member rotation axis when delivering the dose of the drug.

15. A drug delivery device comprising the drive mechanism according to claim 1, further comprising a cartridge, the cartridge holding a plurality of doses of the drug.
